(19) United States
(12) Patent Application Publication
(10) Pub. No.: US 2025/0116647 A1
(43) Pub. Date: Apr. 10, 2025

(54) METHODS OF ASSESSING A CAPROCK IN A GEOLOGIC SEQUENCE FOR CAPROCK DEFECTS

(71) Applicant: Saudi Arabian Oil Company, Dhahran (SA)

(72) Inventors: Martin E. Poitzsch, Northumberland, NH (US); Rajesh Goteti, Houston, TX (US)

(73) Assignee: Saudi Arabian Oil Company, Dhahran (SA)

(21) Appl. No.: 18/482,062

(22) Filed: Oct. 6, 2023

(51) Int. Cl. G01N 33/28; B65G 5/00; E21B 21/08; E21B 49/08

(52) U.S. Cl. CPC G01N 33/2841; B65G 5/005; E21B 49/081; E21B 49/086; G01N 33/2823; E21B 21/085

(57) ABSTRACT

A method of assessing a caprock for caprock defects comprises drilling a first well into a geologic sequence, the geologic sequence comprising a first subsurface formation, the caprock positioned above the first subsurface formation, and a second subsurface formation positioned above the caprock; sampling subsurface fluids of the geologic sequence for helium concentration within the second subsurface formation, within the caprock, and within the first subsurface formation; drilling a second well into the geologic sequence a pre-determined distance away from the first well; sampling the subsurface fluids for the helium concentration within the second subsurface formation, within the caprock, and within the first subsurface formation through the second well; determining whether a deviation exists between the helium concentration at the first well and at the second well, the deviation indicating the caprock defect is present; and halting further drilling into the geologic sequence upon determining the caprock defect is present.

### TECHNICAL FIELD

Embodiments herein generally relate to methods of assessing caprocks in a geologic sequence for caprock defects, and particularly to methods of assessing a caprock for hydrogen storage or sequestration.

### BACKGROUND

Hydrogen has been investigated as an alternative energy source to fossil fuels such as methane in refinery and power plants applications due at least to its relative abundance. However, due to hydrogen's relatively lower volumetric energy density, large amounts of compressed hydrogen gas are needed to replace or supplement fossil fuel and green energy sources. In the case of supplementing green energy sources, variability of demand may also be a concern, as supplemental demand may vary by the cycles of the day (solar), weeks (e.g. from wind), or months (seasonal use in heating).

### SUMMARY

Accordingly, methods are desired for storage of large quantities of hydrogen that may respond to variabilities in demand. One such method for storage of hydrogen involves geologic sequestration. Particularly, methane has previously been injected into depleted gas reservoirs or saline aquifers for seasonal extraction and use, as these geologic sequences include trapping mechanisms, such as caprocks, that resulted in the storage of the fluids for geologically significant timeframes in the first place. These geologic sequences also have the advantage of large storage capacity for injected fluids. Sequestration of carbon dioxide into the same has also been investigated to reduce greenhouse gas accumulation in the atmosphere.

However, subsurface storage or sequestration of hydrogen poses a number of additional problems compared to the storage or sequestration of methane and/or carbon dioxide. First, hydrogen has a series of incompletely-studied reactions with subsurface fluids as well as the carbonate and/or silica (clay) matrix of the geologic sequence itself. Moreover, hydrogen may also be subject to bacterial degradation, with microbial problems potentially occurring at temperatures below 120° C. or in fluids with salinities below 300 parts per thousand.

Most significant to hydrogen storage or sequestration, however, is the integrity of the trapping mechanism (caprock). Saline aquifers may offer little geological insight into the integrity of their caprock for purposes of storing or sequestering hydrogen, since it has not in fact performed as a gas-confining caprock over geological periods of time. Depleted gas reservoirs do offer geological insight into the storage of methane for geologically significant timeframes, but not directly into the storage of hydrogen. In particular, hydrogen is a smaller molecule with greater penetration ability than methane, meaning that not all methane gas reservoirs may be suitable to store hydrogen.

Accordingly, desired are methods for assessing caprocks in geologic sequences for caprock defects, and particularly for caprock defects that may impact the storage of hydrogen into the subsurface formation. Embodiments herein address the aforementioned need by providing a method to assess caprocks in a geologic sequence for caprock defects by utilizing helium concentration as an indicator for potential hydrogen leakage. Helium has a similar penetration ability due to hydrogen due to their similar size. Quantities of helium, and in particular the helium-4 isotope, may originate in subsurface formations from the radioactive decay of uranium and thorium into alpha particles. These quantities of helium may then gradually migrate upward within geologic sequences until being captured with other gaseous species below a suitable caprock. Accordingly, by assessing caprock-having geologic sequences for helium concentration, an idea of caprock integrity, and thus suitability for hydrogen storage, may be formulated.

Particularly, samples of subsurface fluids may be collected while drilling the one or more wells that will be eventually used to store the hydrogen. Samples and helium concentrations for the same are collected above, within, and below the caprock in question for each well. A subsequent deviation between the wells in helium concentration may be indicative of a caprock defect, rendering the geologic sequence susceptible to hydrogen leakage. Moreover, the drilling of the wells in a geometric pattern may allow the determination of the relative location of the caprock defect, potentially also allowing the remediation of the same. The determination of the caprock defect(s) in such a manner may also prevent unnecessary future capital investment in a geologic sequence no longer exhibiting the potential for hydrogen storage.

In accordance with one embodiment herein, a method of assessing a caprock in a geologic sequence for caprock defects comprises drilling a first well into the geologic sequence, the geologic sequence comprising a first subsurface formation, the caprock positioned above the first subsurface formation, and a second subsurface formation positioned above the caprock; sampling subsurface fluids of the geologic sequence for helium concentration within the second subsurface formation, within the caprock, and within the first subsurface formation; drilling a second well into the geologic sequence a pre-determined distance away from the first well; sampling the subsurface fluids for the helium concentration within the second subsurface formation, within the caprock, and within the first subsurface formation through the second well; determining whether a deviation exists between the helium concentration at the first well and the helium concentration at the second well, the deviation indicating the caprock defect is present; and halting further drilling into the geologic sequence upon determining the caprock defect is present.

In accordance with another embodiment herein, a process of storing hydrogen in a geologic sequence comprises assessing a caprock in the geologic sequence for caprock defects and injecting hydrogen into a first subsurface formation of a first well, the first subsurface formation of a second well, or both upon determining the caprock defect is not present.

### BRIEF DESCRIPTION OF THE DRAWINGS

The following detailed description of specific embodiments herein can be best understood when read in conjunction with the following drawings, where like structure is indicated with like reference numerals and in which:

FIG. 1 illustrates a cross-sectional view of a subsurface formation, as described in embodiments herein;

FIG. 2 illustrates a top-down perspective of the subsurface formation of FIG. 1, as described in embodiments herein;

FIG. 3 illustrates an exemplary plot of helium-4 concentration at a given depth for the first and second wells of FIGS. 1-2, where no deviation indicating a caprock defect is present;

FIG. 4 illustrates an exemplary plot of helium-4 concentration at a given depth for the first and third well of FIGS. 1-2, where a deviation indicating a caprock defect is present;

FIG. 5 illustrates an exemplary plot of helium-4 concentration vs. depth for the first, second, and third wells of FIGS. 1-2, where a deviation indicating a caprock defect is present;

FIG. 6 illustrates a formation fluid sampler as described in embodiments herein;

FIG. 7A illustrates a buoyancy-conveyed fluid sampler being conveyed into one of the wells, as described in embodiments herein;

FIG. 7B illustrates the buoyancy-conveyed fluid sampler of FIG. 8A collecting subsurface fluid, as described in embodiments herein;

FIG. 7C illustrates the buoyancy-conveyed fluid sampler of FIGS. 8A and 8B removing a ballast material, as described in embodiments herein; and

FIG. 8 illustrates an underbalanced drilling fluid system for collecting subsurface fluid samples, as described in embodiments herein.

These and other aspects of the present methods are described in further detail below with reference to the accompanying figures, in which one or more illustrated embodiments and/or arrangements of the systems and methods are shown. In the description of the embodiments that follows, like numerals denote like components across the various figures. The systems and methods of the present application are not limited in any way to the illustrated embodiments and/or arrangements. It should be understood that the systems and methods as shown in the accompanying figures are merely exemplary of the systems and methods of the present application, which can be embodied in various forms as appreciated by one skilled in the art. Therefore, it is to be understood that any structural and functional details disclosed herein are not to be interpreted as limiting the present systems and methods, but rather are provided as a representative embodiment and/or arrangement for teaching one skilled in the art one or more ways to implement the present systems and methods.

### DETAILED DESCRIPTION

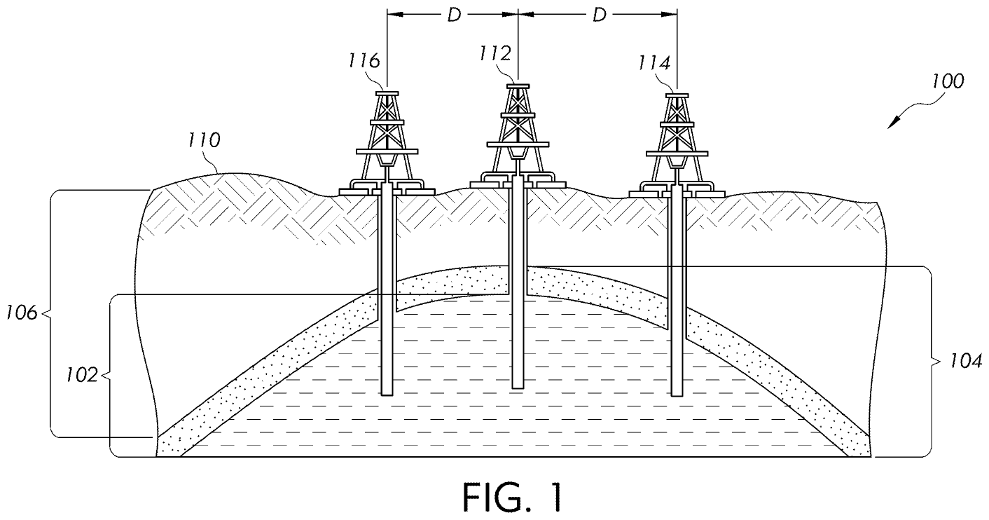

Embodiments herein generally relate to methods of assessing a caprock in a geologic sequence for caprock defects, and particularly to methods for assessing a caprock for hydrogen storage or sequestration. The methods and systems herein are described, in some instances, in the context of the geologic sequence of FIG. 1. However, it should be understood that the methods and systems described herein may have applicability with other geologic sequences than are illustrated in FIG. 1, as would be appreciated by those skilled in the art.

As used herein, the terms “downhole” and “uphole” may refer to a position within a wellbore relative to the surface, with uphole indicating direction or position closer to the surface and downhole referring to direction or position farther away from the surface. Similarly, as used herein, the terms “downward” and “upward” may refer to a position within a subterranean environment or geologic sequence relative to the surface, with upward indicating direction or position closer to the surface and downward referring to direction or position farther away from the surface.

As used herein, a “subsurface formation” may refer to a body of rock that is sufficiently distinctive and continuous from the surrounding rock bodies of the geologic sequence, such that the body of the rock may be mapped as a distinct entity. A subsurface formation is, therefore, sufficiently homogenous to form a single identifiable unit containing similar properties throughout the subsurface formation, including, but not limited to, porosity and permeability.

As used herein, “wellbore,” may refer to a drilled hole or borehole extending from the surface of the Earth down to the geologic sequence, including the openhole or unlined portion. The wellbore may form a pathway capable of permitting fluids to traverse between the surface and the geologic sequence. The wellbore may include at least a portion of a fluid conduit that links the interior of the wellbore to the surface. The fluid conduit connecting the interior of the wellbore to the surface may be capable of permitting regulated fluid flow from the interior of the wellbore to the surface and may permit access between equipment on the surface and the interior of the wellbore.

As used herein, a “wellbore wall” may refer to the interface through which fluid may transition between the geologic sequence and the interior of the wellbore. The wellbore wall may be unlined (that is, bare rock or formation) to permit such interaction with the geologic sequence or lined, such as by a tubular string, to prevent such interactions. The wellbore wall may also define the void volume of the wellbore.

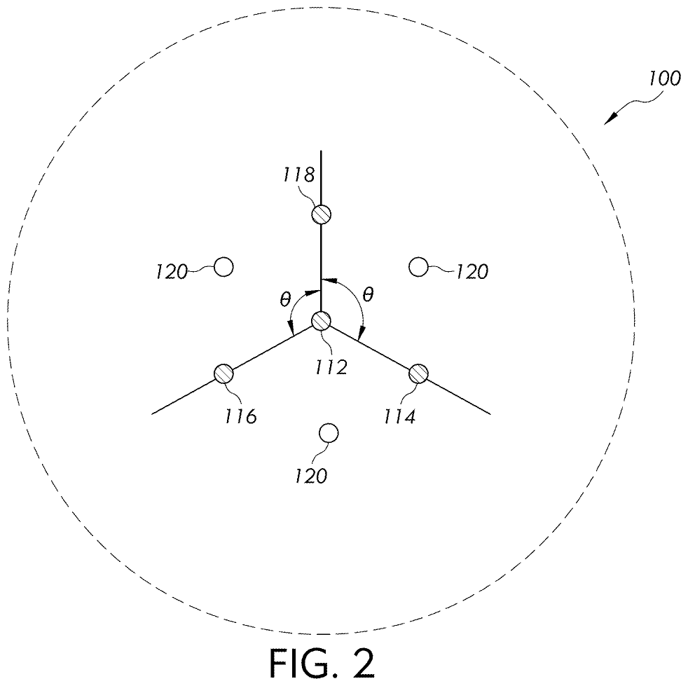

Referring now to FIGS. 1 and 2, a method of assessing a caprock 104 in a geologic sequence 100 may comprise drilling a first well 112 into the geologic sequence 100. As shown in FIG. 1, the geologic sequence 100 may comprise a first subsurface formation 102, the caprock 104 positioned above the first subsurface formation 102, and a second subsurface formation 106 positioned above the caprock 104. As also shown in FIG. 1, the geologic sequence 100 may take the form of an anticline. Accordingly, as shown in FIGS. 1 and 2, the first well 112 may be radially centered on a highest point of the caprock 104, i.e. at the top of the anticline. As explained in further detail hereinbelow, and without being limited by theory, the positioning of the first well 112 on the highest point of the caprock 104 may permit the drilling of additional wells in a drilling grid that allows the proportional assessment of the caprock 104.

Still referring to FIG. 1, the method may then comprise sampling subsurface fluids of the geologic sequence 100 for helium concentration within the second subsurface formation 106, within the caprock 104, and within the first subsurface formation 102. Without being limited by theory, the sampling of the subsurface fluids may occur after or concurrently with the drilling of each well, as described in further detail hereinbelow. Further, the sampling of the subsurface fluid may occur in an unlined portion of each drilled well.

Still referring to FIG. 1, the method may then comprise drilling a second well into the geologic sequence 100 a pre-determined distance “D” away from the first well 112. The predetermined distance may be determined according to the experience of personnel including but not limited to such factors as first subsurface formation 102 temperature, pressure, permeability, porosity, and surface area. The method may then comprise sampling the subsurface fluids for the helium concentration within the second subsurface formation 106, within the caprock 104, and within the first subsurface formation 102 through the second well.

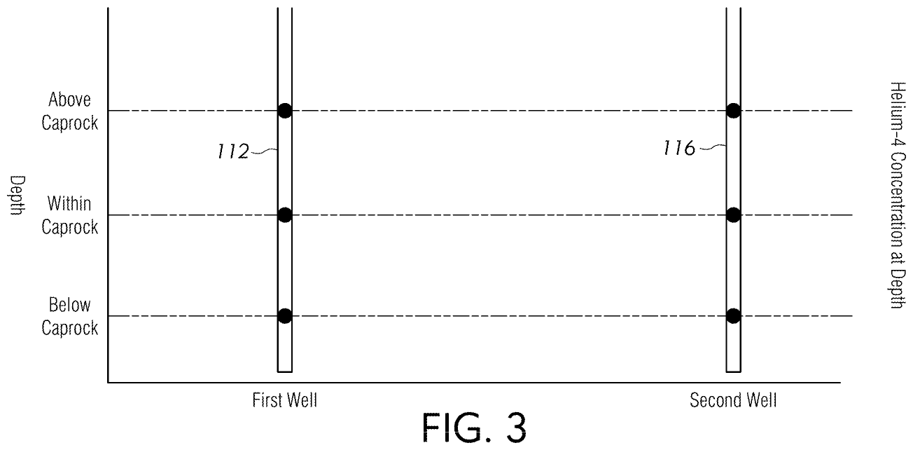

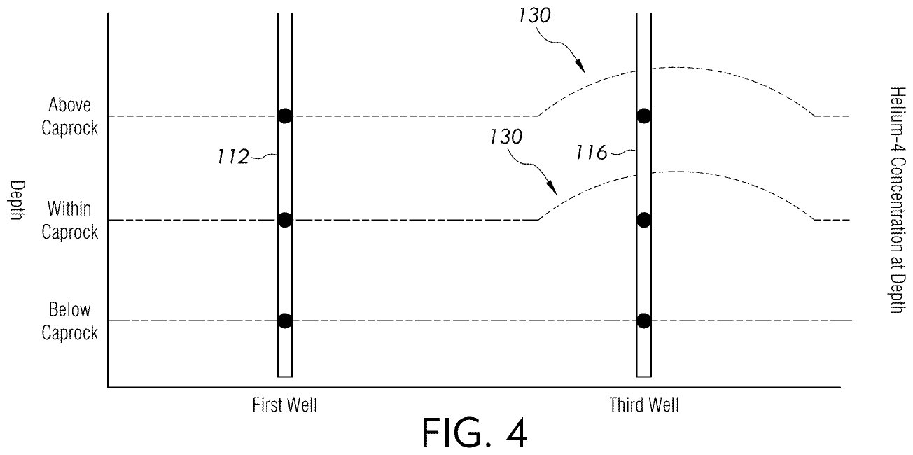

The method may then comprise determining whether a deviation 130 exists between the helium concentration at the first well 112 and the helium concentration at the second well, the deviation 130 indicating the caprock defect is present, such as somewhere between the two wells being compared. For example, and as illustrated in FIGS. 3 and 4, flat contours between the wells being compared would tend to show that no deviation 130, and thus no caprock defect is present between the two wells, as shown in FIG. 3. However, the observation of a sloped or stepped increase in concentration between the two wells not reflected to the same degree of magnitude below the caprock 104 (to avoid false positives in a localized area with higher helium), such as that illustrated in FIG. 4, would tend to show that the deviation 130, and thus the caprock defect, is present between the two wells.

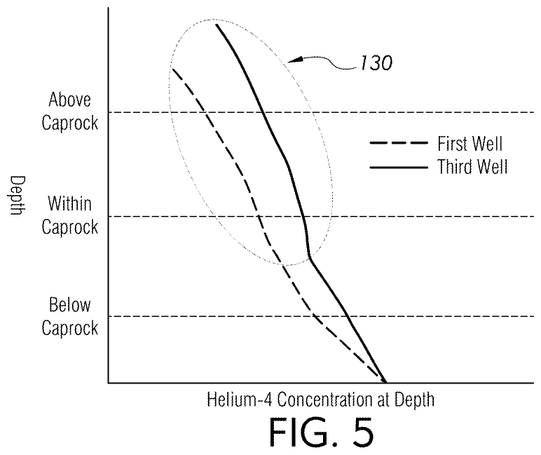

Similarly, and now referring to FIG. 5, the deviation 130 may be indicated on a vertical plot of helium concentration vs. depth for each of the wells measured. For example, and in embodiments, if the helium concentrations vs. depth for the wells are not relatively parallel it may indicate the deviation 130 indicating the caprock defect is present, such as somewhere between the two wells being measured. Likewise, if the helium concentration vs. depth for the wells is relatively parallel, it may be an indication that the caprock defect is not present, even if the helium concentration is on average greater than or less than the initial well.

Upon determining that the caprock defect is present, the method may further comprise halting further drilling into the geologic sequence 100. If the deviation 130 does not exist, drilling may continue as there is potentially no caprock defect present between the two wells.

Referring to FIG. 2, illustrated is a top-down view of the geologic sequence 100 of FIG. 1. As shown in FIGS. 1-2, upon determining the caprock defect is not present between the first and second wells, the method may further comprise drilling a third well 116 into the geologic sequence 100. As shown in FIG. 1, the third well 116 may be drilled at or approximately at the pre-determined distance away from the first well 112, similar to the second well. Further, as illustrated in FIG. 2, the third well 116 may be positioned at an angle “θ”, such as at 110° to 130° or at approximately 120°, from a line defined from the first well 112 to the second well.

The method may then comprise sampling the subsurface fluids for the helium concentration within the second subsurface formation 106, within the caprock 104, and within the first subsurface formation 102 through the third well 116. Similar to the first and second wells, the method may then further comprise determining whether the deviation 130 exists between the helium concentration at the first well 112 and the helium concentration at the third well 116. Upon determining the caprock defect is present between the first well 112 and the third well 116, further drilling may be halted into the geologic sequence 100. However, if the deviation 130 is not present, drilling may continue as there is potentially no caprock defect present between the wells.

Still referring to FIGS. 1-2, upon determining the caprock defect is not present in the first through third wells, the method may further comprise drilling a fourth well 118 into the geologic sequence 100. The fourth well 118 may be drilled the pre-determined distance away from the first well 112, similar to the second and third wells. Further, as illustrated in FIG. 2, the fourth well 118 may be positioned at the angle “θ”, from a line defined from the first well 112 to the second well, and from a second line defined from the first well 112 to the third well 116.

The method may then comprise sampling the subsurface fluids for the helium concentration within the second subsurface formation 106, within the caprock 104, and within the first subsurface formation 102 through the fourth well 118. Similar to the first through third wells, the method may then further comprise determining whether the deviation 130 exists between the helium concentration at the first well 112 and the helium concentration at the fourth well 118. Upon determining the deviation 130, and thus the caprock defect, is present between the first well 112 and the fourth well 118, further drilling may be halted into the geologic sequence 100. However, if the deviation 130 does not exist, drilling may continue as there is potentially no caprock defect present between the wells.

Still referring to FIGS. 1-2, upon determining the caprock defect is not present in the first through third wells, the method may further comprise drilling a fourth well 118 into the geologic sequence 100. The fourth well 118 may be drilled the pre-determined distance away from the first well 112, similar to the second and third wells. Further, as illustrated in FIG. 2, the fourth well 118 may be positioned at the angle “θ”, from a line defined from the first well 112 to the second well, and from a second line defined from the first well 112 to the third well 116.

The method may then comprise sampling the subsurface fluids for the helium concentration within the second subsurface formation 106, within the caprock 104, and within the first subsurface formation 102 through the fourth well 118. Similar to the first through third wells, the method may then further comprise determining whether the deviation 130 exists between the helium concentration at the first well 112 and the helium concentration at the fourth well 118. Upon determining the deviation 130, and thus the caprock defect, is present between the first well 112 and the fourth well 118, further drilling may be halted into the geologic sequence 100. However, if the deviation 130 is not present, drilling may continue as there is potentially no caprock defect present between the wells.

Still referring to FIGS. 1-2, upon determining the caprock defect is not present in the first through fourth wells, the method may further comprise drilling at least one additional well 120 into the geologic sequence 100. The at least one additional well 120 may be positioned between any pair of the first through fourth wells, as shown in FIG. 2. The at least one additional well 120 may also be drilled the pre-determined distance away from the first well 112, although this is not required. Further, while FIG. 2 illustrates a hexagonal drilling grid, it is contemplated that any polygonal shape of drilling grid may be used, such as a square or rectangular drilling grid, which may or may not be centered around the first well 112. Further, while three of the at least one additional well 120 are illustrated in FIG. 2, it is contemplated that any number of additional wells 120 may be drilled into the formation according to the present embodiments, the decision of which may be based on a number of factors known to one of ordinary skill in the art, such as, but not limited to, area of the geologic sequence 100, permeability, porosity, pressure of the first subsurface formation 102, etc.

After drilling the at least one additional well 120, the method may then comprise sampling the subsurface fluids for the helium concentration within the second subsurface formation 106, within the caprock 104, and within the first subsurface formation 102 through the at least one additional well 120. Similar to the first through fourth wells, the method may then further comprise determining whether the deviation 130 exists between the helium concentration at the first well 112 and the helium concentration at the at least one additional well 120. Upon determining the deviation 130, and thus the caprock defect, is present between the first well 112 and the at least one additional well 120, further drilling may be halted into the geologic sequence 100. However, if the deviation 130 is not present, drilling may continue as there is potentially no caprock defect present between the wells.

In such a manner, wells may be drilled into the geologic sequence 100 using the above pattern until a sufficient area of the caprock 104 has been assessed to determine suitability for storage of hydrogen. Accordingly, and as previously stated, embodiments herein are also directed to processes of storing or sequestering hydrogen in the geologic sequence 100. The process may initially comprise assessing the caprock 104 in the geologic sequence 100 for the caprock defects according to any of the methods hereinbefore described. Upon determining the caprock defect is not present, the process may further comprise injecting hydrogen into the first subsurface formation 102 of one or more of the first well 112, the second well, the third well 116, the fourth well 118, and the at least one additional well 120.

As previously stated, embodiments of the method herein may comprise sampling the subsurface fluids of the geologic sequence 100 for helium concentration. The sampling of the subsurface fluids may occur within the second subsurface formation 106, within the caprock 104, and within the first subsurface formation 102 for each drilled well. However, the sampling may also occur at multiple points within the previous three intervals. For example, multiple subsurface fluid samples may be collected within the second subsurface formation 106, within the caprock 104, and within the first subsurface formation 102 at varying depths. In embodiments, sampling the subsurface fluids of the geologic sequence 100 may also occur in an unlined portion of each well, such as prior to cementing the portion to be sampled.

As previously stated, hydrogen has a series of incompletely-studied reactions with subsurface fluids as well as the carbonate and/or silica (clay) matrix of common geologic sequences. Moreover, hydrogen may also be subject to bacterial degradation in certain conditions, with microbial problems potentially occurring at temperatures below 120° C. or in fluids with salinities below 300 parts per thousand. Accordingly, subsurface fluids in the methods and processes hereinbefore described may comprise a salinity of greater than or equal to 300 parts per thousand (ppt); a temperature of greater than or equal to 120° C.; or both.

As previously stated, sampling of the subsurface fluids may occur within the second subsurface formation 106, within the caprock 104, and within the first subsurface formation 102 for each drilled well. Without being limited by theory, it may be advantageous to sample the subsurface fluids at fixed distances above, within, or below the caprock 104 to permit analogous measurements for helium concentration across the geologic sequence 100. For example, and in embodiments, sampling the subsurface fluid within the second subsurface formation 106 may occur at a pre-defined vertical depth, such as from 1 foot to 30 feet or from 10 feet to 30 feet, above the caprock 104. Further, sampling the subsurface fluid within the caprock 104 may occur at a pre-defined vertical depth, such as from 1 foot to 30 feet, within the caprock 104. Further yet, sampling the subsurface fluid within the first subsurface formation 102 may occur at a pre-defined vertical depth, such as from 1 foot to 30 feet, below the caprock 104.

Without being limited by theory, the pre-defined vertical depth above, below, or within the caprock 104 may be chosen according to the experience of personnel, such as according to the total thickness of the second subsurface formation 106, the caprock 104, or the first subsurface formation 102. The vertical depth to the first subsurface formation 102, the caprock 104, and the second subsurface formation 106, as well as the pre-defined vertical depths above, below, or within the caprock 104, may be determined utilizing one or more subsurface formation analysis methods, including but not limited to well logging, surface seismography, drill cuttings analysis, or combinations thereof, as would be understood by one of ordinary skill in the art.

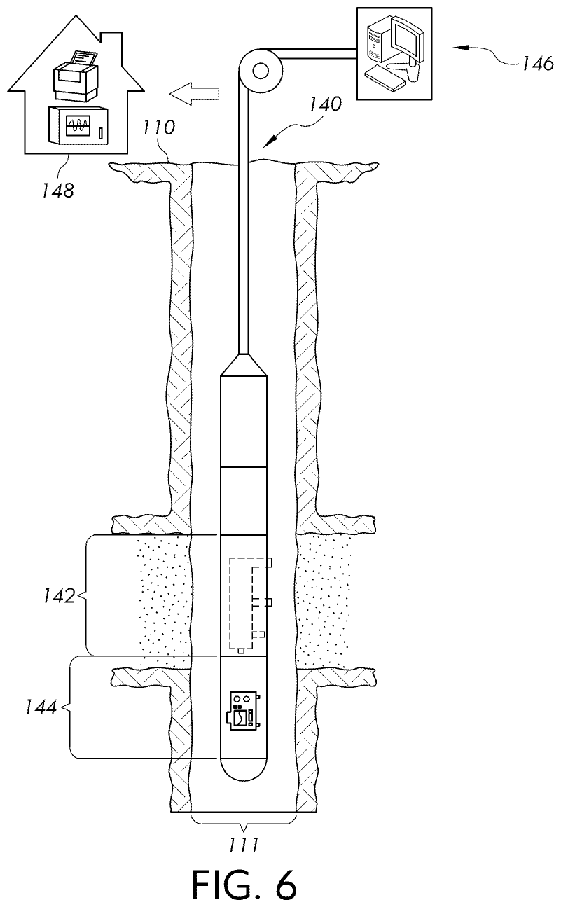

Now referring to FIGS. 6-8, and as previously stated, sampling of the subsurface fluids may occur within the second subsurface formation 106, within the caprock 104, and within the first subsurface formation 102 for each well. The sampling may occur using varied tools and systems, as explained in further detail below.

For example, and as illustrated in FIG. 6, subsurface fluid sampling may occur utilizing a subsurface fluid sampler 140. The subsurface fluid sampler 140 may comprise a wireline subsurface fluid sampler 140, including but not limited to Schlumberger's MDT®, Halliburton's RDT®, Weatherford's RES®, or any similar wireline measurement tool known in the art. The subsurface fluid sampler 140 may alternatively comprise a drill-string subsurface fluid sampler 140, including but not limited to Baker-Hughes FASTrak® HD or Prism products, Halliburton's GeoTap®, Schlumberger's SpectraSphere®, or any similar measurement-while-drilling tool known in the art. Accordingly, sampling the subsurface fluids for the helium concentration within the second subsurface formation 106, within the caprock 104, and within the first subsurface formation 102 may comprise providing the subsurface fluid sampler 140 into one or more of the wells, such as into a wellbore 111 of the one or more wells; collecting subsurface fluid into a fluid chamber 142 of the subsurface fluid sampler 140; and analyzing the subsurface fluid therein for helium concentration.

For example, and in embodiments, the subsurface fluid may be analyzed by recovering the subsurface fluid from the fluid chamber 142 of the subsurface fluid sample 140 and then analyzing the subsurface fluid in an analytical laboratory 148 utilizing a mass spectrometer (MS) or a MS combined with gas chromatograph (GC-MS). The subsurface fluid sampler 140 may also or alternatively comprise one or more fluid analyzing tools 144 for reservoir fluid sampling quality-control, which may include, but may not be limited to, a NIR (near-infrared) spectrometer, a resistivity measurement cell, or both. As shown in FIG. 5, the one or more fluid analyzing tools 144 may also be in communication with a processing unit 146 at or near the surface 110, such as by wireline.

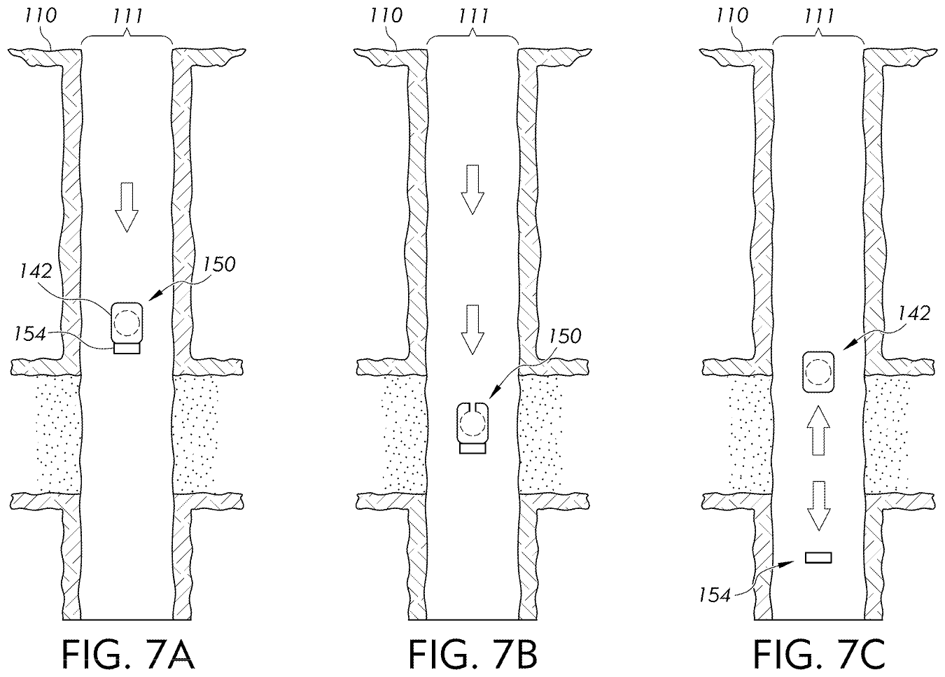

Now referring to FIGS. 7A-7C, subsurface fluid sampling may also or alternatively occur in open-hole wellbores utilizing a buoyancy-conveyed fluid sampler 150, such as Saudi Aramco's SensorBall®. Accordingly, sampling the subsurface fluids for the helium concentration within the second subsurface formation 106, within the caprock 104, and within the first subsurface formation 102 may comprise providing the buoyancy-conveyed fluid sampler 150 into one or more of the wells, such as into the wellbore 111 of the wells, as illustrated in FIG. 7A; collecting the subsurface fluid into the fluid chamber 142 of the buoyancy-conveyed fluid sampler 150, as illustrated in FIG. 7B; removing a ballast material 154 from the buoyancy-conveyed fluid sampler 150, as illustrated in FIG. 7C, to return the buoyancy-conveyed fluid sampler 150 to a surface 110 of one or more wells; and analyzing the subsurface fluid therein captured for helium concentration in a surface analytical laboratory using a mass spectrometer (MS) or a MS combined with gas chromatograph (GC-MS). In embodiments, the ballast material 154 may be removed by magnetically decoupling the ballast material 154 from the fluid chamber 142 of the buoyancy-conveyed fluid sampler 150. The subsurface fluid collected into the fluid chamber 142 of the buoyancy-conveyed fluid sampler may be analyzed in a similar manner to that for the subsurface fluid collected in the subsurface fluid sampler 140.

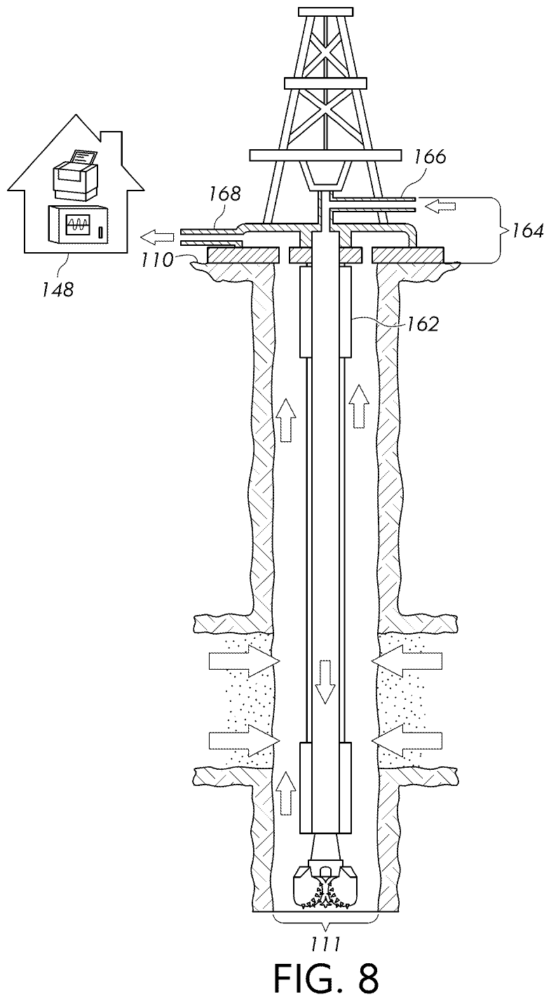

Now referring to FIG. 8, subsurface fluid sampling may occur utilizing an underbalanced drilling fluid system concurrently with the drilling of one or more of the wells. As used herein, an “underbalanced drilling fluid system” refers to a drilling procedure wherein the overbearing fluid pressure of the drilling fluid in the wellbore 111 of the well is maintained below the fluid pressure of the geologic sequence 100. The result of an underbalanced drilling fluid system is the influx of subsurface fluids from the geologic sequence 100 into the wellbore 111 during the drilling of the well, as well as the subsequent circulation of the subsurface fluids to the surface 110 where they may either be separated, analyzed, or both. Accordingly, sampling the subsurface fluids for the helium concentration within the second subsurface formation 106, within the caprock 104, and within the first subsurface formation 102 may additionally or alternatively comprise circulating the underbalanced drilling fluid through a drill string 162 within one or more of the wells during the drilling of the same; collecting the subsurface fluid along with the underbalanced drilling fluid at the surface 110 of the one or more wells; and analyzing the subsurface fluid at the surface 110. The subsurface fluid may be analyzed using a mass spectrometer (MS) or a MS combined with gas chromatograph (GC-MS), such as in the analytical laboratory 148, such as a mud logging cabin or the like. In embodiments including the underbalanced drilling fluid, the one or more wells may further comprise a pressurized wellhead 164 positioned at the surface 110 of the one or more wells, injection lines 166 upstream of the drill string 162, and outlet lines 168 downstream of the drill string 162.

As previously stated, the subsurface fluids may be sampled for the helium concentration within the second subsurface formation 106, within the caprock 104, and within the first subsurface formation 102. In embodiments, the helium concentration may comprise the mass percent of helium-4 isotope in the subsurface fluid. However, the helium concentration may additionally or alternatively comprise the mass ratio of helium-3 isotope to the helium-4 isotope in the subsurface fluid. Without being limited by theory, the mass ratio of helium-3 isotope to the helium-4 isotope may give insight into the geochemical origin of helium in the geologic sequence. Helium-3 isotope is primarily understood as a primordial isotope in origin, coming up from the deepest regions of the earth's core and mantle, where it was accreted from the solar nebula at the earliest time of planetary formation, i.e., 4.5 billion years ago. Helium-4 isotope, on the other hand, formed from alpha particles emitted by uranium and thorium decaying over billions of years and could form wherever those elements were deposited in various strata of the earth, including not only the deepest regions, but also the upper mantle and crust; and in both ancient basaltic or granitic structures, such as plates or mountain ranges, but also eroded volcanic sediments deposited into shales or reservoirs at all possible depths. Thus, much of the helium-4 isotope could come up from very deep in the earth; but contributions could also form even within a shallow reservoir, and the isotopic ratio could vary measurably across such a reservoir.

Accordingly, without being limited by theory, a relatively large mass ratio of helium-3 isotope to helium-4 isotope may be indicative that the helium in the subsurface fluid migrated from upper mantle and deeper areas within the Earth, rather than being formed in the geologic sequence. Comparing the mass ratio of helium-3 isotope to helium-4 isotope across the wells of the geologic sequence may also provide further insight into the relative heterogeneity of the geologic sequence and first subsurface formation as well as the fluid interconnectivity throughout. In other words, without being limited by theory, a first area in the first subsurface formation where the mass ratio of helium-3 isotope to helium-4 isotope is relatively less than a second area may indicate that the first area has a lesser degree of fluid interconnectivity, as helium-3 isotope originating from subsurface fluid migration is of a lesser concentration than helium-4 isotopes that may generate within the first subsurface formation. Consequently, the first area may also have less capability for storage of volumetrically significant amounts of hydrogen. Accordingly, the mass ratio of helium-3 isotope to the helium-4 isotope in the subsurface fluid may also be used to determine the ideal areas in which to begin injection of the hydrogen into the first subsurface formation so as to allow equal distribution of the injected hydrogen across the first subsurface formation of the geologic sequence.

It is noted that recitations herein of a component herein being “operable” or “sufficient” in a particular way, to embody a particular property, or to function in a particular manner, are structural recitations, as opposed to recitations of intended use. More specifically, the references herein directed to the manner in which a component is “operable” or “sufficient” denotes an existing physical condition of the component and, as such, is to be taken as a definite recitation of the structural characteristics of the component.

The singular forms “a,” “an” and “the” include plural referents, unless the context clearly dictates otherwise.

Herein, ranges are provided. It is envisioned that each discrete value encompassed by the ranges are also included. Additionally, the ranges which may be formed by each discrete value encompassed by the explicitly disclosed ranges are equally envisioned.

As used herein and in the appended claims, the words “comprise,” “has,” and “include” and all grammatical variations thereof are each intended to have an open, non-limiting meaning that does not exclude additional elements or steps.

As used herein, terms such as “first” and “second” are arbitrarily assigned and are merely intended to differentiate between two or more instances or components. It is to be understood that the words “first” and “second” serve no other purpose and are not part of the name or description of the component, nor do they necessarily define a relative location, position, or order of the component. Furthermore, it is to be understood that the mere use of the term “first” and “second” does not require that there be any “third” component, although that possibility is contemplated under the scope herein.

Having described the subject matter herein in detail and by reference to specific embodiments, it is noted that the various details disclosed herein should not be taken to imply that these details relate to elements that are essential components of the various embodiments described herein. Further, it will be apparent that modifications and variations are possible without departing from the scope herein, including, but not limited to, embodiments defined in the appended claims.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

What is claimed is:

1. A method of assessing a caprock in a geologic sequence for caprock defects, the method comprising:
drilling a first well into the geologic sequence, the geologic sequence comprising a first subsurface formation, the caprock positioned above the first subsurface formation, and a second subsurface formation positioned above the caprock;
sampling subsurface fluids of the geologic sequence for helium concentration within the second subsurface formation, within the caprock, and within the first subsurface formation;
drilling a second well into the geologic sequence a pre-determined distance away from the first well;
sampling the subsurface fluids for the helium concentration within the second subsurface formation, within the caprock, and within the first subsurface formation through the second well;
determining whether a deviation exists between the helium concentration at the first well and the helium concentration at the second well, the deviation indicating the caprock defect is present; and
halting further drilling into the geologic sequence upon determining the caprock defect is present.

2. The method of claim 1, further comprising:
drilling a third well into the geologic sequence the pre-determined distance away from the first well upon determining the caprock defect is not present, wherein the third well is positioned at an approximate 120 degree angle from a line defined from the first well to the second well;
sampling the subsurface fluids for the helium concentration within the second subsurface formation, within the caprock, and within the first subsurface formation through the third well;
determining whether the deviation exists between the helium concentration at the first well and the helium concentration at the third well;
halting further drilling into the geologic sequence upon determining the caprock defect is present.

3. The method of claim 2, further comprising:
drilling a fourth well into the geologic sequence the pre-determined distance away from the first well upon determining the caprock defect is not present, wherein the fourth well is positioned at an approximate 120 degree angle from the line defined from the first well to the second well, and at an approximate 120 degree angle from a second line defined from the first well to the third well;
sampling the subsurface fluids for the helium concentration within the second subsurface formation, within the caprock, and within the first subsurface formation through the fourth well;
determining whether the deviation exists between the helium concentration at the first well and the helium concentration at the fourth well;
halting further drilling into the geologic sequence upon determining the caprock defect is present.

4. The method of claim 3, further comprising:
drilling at least one additional well into the geologic sequence upon determining the caprock defect is not present;
sampling the subsurface fluids for helium concentration within the second subsurface formation, within the caprock, and within the first subsurface formation through the at least one additional well;
determining whether the deviation exists between the helium concentration at the first well and the at least one additional well;
halting further drilling into the geologic sequence upon determining the caprock defect is present.

5. The method of claim 1, wherein sampling the subsurface fluids for the helium concentration within the second subsurface formation, within the caprock, and within the first subsurface formation comprises:
providing a subsurface fluid sampler into the first well, the second well or both;
collecting subsurface fluid into a fluid chamber of the subsurface fluid sampler; and
analyzing the subsurface fluid in the fluid chamber for helium concentration.

6. The method of claim 1, wherein the formation fluid sampler is a wireline subsurface fluid sampler or a drill-string subsurface fluid sampler.

7. The method of claim 1, wherein sampling the subsurface fluids for the helium concentration within the second subsurface formation, within the caprock, and within the first subsurface formation comprises:
providing a buoyancy-conveyed fluid sampler into the first well, the second well, or both;
collecting the subsurface fluid into a fluid chamber of the buoyancy-conveyed fluid sampler;
removing a ballast material from the buoyancy-conveyed fluid sampler to return the buoyancy-conveyed fluid sampler to a surface of the first well, the second well, or both; and
analyzing the subsurface fluid in the fluid chamber for helium concentration.

8. The method of claim 1, wherein sampling the subsurface fluids for the helium concentration within the second subsurface formation, within the caprock, and within the first subsurface formation comprises:
circulating an underbalanced drilling fluid through a drill-string within the first well, the second well, or both during the drilling of the first well, the second well, or both;
collecting the subsurface fluid along with the underbalanced drilling fluid at a surface of the first well, the second well, or both; and
analyzing the subsurface fluid for helium concentration at the surface, and wherein the first well, the second well, or both comprise
a pressurized wellhead positioned at the surface of the first well, the second well, or both,
injection lines upstream of the drill-string, and
outlet lines downstream of the drill string.

9. The method of claim 1, wherein sampling the subsurface fluids for the helium concentration within the second subsurface formation, within the caprock, and within the first subsurface formation occurs in an unlined portion of the first well, the second well, or both.

10. The method of claim 1, wherein a vertical depth to the first subsurface formation, the caprock, and the second subsurface formation in the first well, the second well, or both is determined utilizing well logging, surface seismography, drill cuttings analysis, or combinations thereof.

11. The method of claim 10, wherein sampling the subsurface fluid within the second subsurface formation occurs at a pre-defined vertical depth above the caprock.

12. The method of claim 10, wherein sampling the subsurface fluid within the first subsurface formation occurs at a pre-defined vertical depth below the caprock.

13. The method of claim 10, wherein sampling the subsurface fluid within the caprock occurs at a pre-defined vertical depth within the caprock.

14. The method of claim 10, wherein the helium concentration comprises the mass percent of helium-4 isotope in the subsurface fluid.

15. The method of claim 14, wherein the helium concentration further comprises the mass ratio of helium-3 isotope to the helium-4 isotope in the subsurface fluid.

16. The method of claim 1, wherein the subsurface fluids comprise:
a salinity of greater than or equal to 300 parts per thousand (ppt);
a temperature of greater than or equal to 120° C.; or
both.

17. The method of claim 1, wherein sampling of the subsurface fluids of the geologic sequence for helium concentration within the first well, the second well, or both, occurs at multiple points within the second subsurface formation, at multiple points within the caprock, at multiple points within the first subsurface formation, or combinations thereof.

18. The method of claim 1, wherein the first well is radially centered on a highest point of the caprock.

19. A process of storing hydrogen in a geologic sequence, the process comprising:
assessing a caprock in the geologic sequence for caprock defects according to claim 1; and
injecting hydrogen into the first subsurface formation of the first well, the first subsurface formation of the second well, or both upon determining the caprock defect is not present.

20. A process of storing hydrogen in a geologic sequence, the process comprising:
assessing a caprock in the geologic sequence for caprock defects according to claim 4; and
injecting hydrogen into the first well, the second well, the third well, the at least one additional well, or combinations thereof upon determining the caprock defect is not present.
